(19) United States
(12) Patent Application Publication
(10) Pub. No.: US 2002/0044966 A1
(43) Pub. Date: Apr. 18, 2002

(54) Pharmaceutical formulations containing an opioid and an alpha-agonist

(76) Inventors: Johannes Bartholomaeus, Aachen (DE); Juergen Betzing, Loerrach-Tumringen (DE)

Correspondence Address: CROWELL & MORING LLP, INTELLECTUAL PROPERTY GROUP, P.O. BOX 14300, WASHINGTON, DC, 20044-4300, US

(21) Appl. No.: 09/907,447

(22) Filed: Jul. 18, 2001

Related U.S. Application Data
(63) Continuation of application No. PCT/EP00/00318, filed on Jan. 17, 2000.

(30) Foreign Application Priority Data
Jan. 18, 1999 (DE) 199 01 684.4

(51) Int. Cl. A61K 9/22; A61K 31/485

(52) U.S. Cl. 424/468; 514/282

(57) ABSTRACT

Pharmaceutical formulations containing an opioid, an α-agonist and/or their physically compatible salts, from which at least one medicinally active ingredient is released in a sustained manner.

### BACKGROUND OF THE INVENTION

This invention relates to pharmaceutical formulations containing an opioid, an α-agonist and/or in each case the physiologically compatible salt thereof, from which formulations at least one pharmaceutical active substance is released in delayed manner.

Due to their strong analgesic action, opioids are used to alleviate moderately severe and severe acute pain. One major disadvantage of using opioids, however, resides in the severe side-effects associated therewith. Side-effects on the gastrointestinal tract, such as for example severe constipation, frequently occur. They moreover cause respiratory depression and, on repeated administration, dependency, which may result in abuse. A further disadvantage is the rapid development of tolerance.

It is known to administer opioids and α-agonists as individual preparations using various pharmaceutical formulations. In addition to known non-controlled release systems, there are also controlled release systems with opioids, such as described in WO 95/14460 or EP-A-0 647 448, in which, inter alia, butyrates, ketobemidone, codeine and the like are used. EP-B-0 271 193 discloses a controlled release system using solely hydromorphone. Controlled release systems with α-agonists are disclosed in EP-A-0 805 677 or U.S. Pat. No. 5,484,607. In both cases, clonidine is used as the only α-agonist.

### SUMMARY OF THE INVENTION

It is the object of the present invention to provide a pharmaceutical formulation which is suitable for treating severe to very severe pain.

Another object of the invention is to provide an effective pain relieving pharmaceutical formulation which does not exhibit the typical side-effects of opioids.

It is also an object of the invention to provide an effective pain relieving pharmaceutical formulation which very considerably delays or completely prevents the development of opioid tolerance.

These and other objects are achieved in accordance with the present invention by providing pharmaceutical formulations which contain an opioid, an α-agonist and/or in each case physiologically compatible salts thereof, from which formulations at least one pharmaceutical active substance is released in delayed manner.

It is preferably the opioid which is released from the pharmaceutical formulation according to the invention in delayed manner. Delayed release of the opioid preferably proceeds over a period of 8 hours, particularly preferably over 12 hours and especially preferably over 24 hours.

It is likewise preferred for both the pharmaceutical active substances to be released from the pharmaceutical formulation according to the invention in delayed manner.

The pharmaceutical formulation according to the invention preferably contains morphine, hydromorphone, codeine, oxycodone, dihydrocodeine, dextropropoxyphene, buprenorphine, levomethadone, fentanyl, sufentanil, etorphine, pentazocine, tilidine, tramadol, levorphanol, methadone, dihydromorphine, pethidine, piritramide or a physiologically compatible salt of any of the stated opioids as the opioid. The pharmaceutical formulation according to the invention particularly preferably contains morphine, tramadol and/or a physiologically compatible salt thereof as the opioid.

The pharmaceutical formulation according to the invention preferably contains clonidine, guanfacine, guanabenz, lofexidine, adrenaline, methyldopa, noradrenaline, methoxamine, oxymetazoline, xylometazoline, teryzoline, ST-91, medetomidine, dexmedetomidine, agmatine, UK14, 304, para-aminoclonidine, U-47, 476A, DJ-741, ICI-106270, xylazine, talipexole (BHT-920), naphazoline, tizanidine and/or a physiologically compatible salt of the stated α-agonists as the α-agonist. The pharmaceutical formulation according to the invention particularly preferably contains clonidine, guanfacine and/or a physiologically compatible salt thereof as the α-agonist.

Especially preferably, the pharmaceutical formulation according to the invention contains morphine and/or tramadol as the opioid and clonidine as the α-agonist and/or in each case the physiologically compatible salt thereof.

Physiologically compatible salts of the active substances which are preferably used include acetates, tartrates, sulfates, hydrochlorides, phosphates and additionally salicylates and acetylsalicylates for the group of opioids.

The weight ratio of the opioid to the α-agonist in the pharmaceutical formulations according to the invention is preferably 200:1 to 10:1. In a particularly preferred embodiment, the weight ratio of the opioid to the α-agonist is 100:1 to 10:1.

The pharmaceutical formulation according to the invention is preferably administered orally. Preferred oral pharmaceutical formulations include tablets, sugar-coated tablets, and capsules. It is particularly preferred to administer tablets and very particularly preferred to administer multilayer tablets.

The pharmaceutical formulation according to the invention may also be in multiparticulate form, such as for example in the form of microtablets, microcapsules, ion exchange resinates, granules, active substance crystals or pellets. The pharmaceutical formulation according to the invention may preferably also assume the form of a pellet tablet which disintegrates particularly quickly.

Controlled release of the respective active substances may preferably be achieved by a controlled release coating, immobilization on an ion exchange resin, embedding in a controlled release matrix, or a combination thereof.

Controlled release is preferably achieved by means of controlled release coatings. Suitable controlled release coatings include water-insoluble waxes or polymers, such as for example acrylic resins, preferably poly(meth)acrylates, or water-insoluble celluloses, preferably ethylcellulose.

These materials are known from the prior art; see, for example Bauer, Lehmann, Osterwald, Rothgang, “Überzogene Arzneiformen”, Wissenschaftliche Verlagsgesellschaft mbH, Stuttgart, 1988, pp. 69 et seq.

In order to regulate the rate of release of the active substance, the controlled release coatings may also contain, in addition to the water-insoluble polymers, quantities of up to 30 wt. % of preferably water-soluble polymers which do not delay release, such as polyvinylpyrrolidone or water-soluble celluloses, preferably hydroxypropylmethylcellulose or hydroxypropylcellulose, and/or hydrophilic pore formers, such as sucrose, sodium chloride or mannitol and/or known plasticizers.

Another conventional method for achieving controlled release is immobilization of the active substances on ion exchange resins. Colestyramine is preferably used as an anionic ion exchange resin, while polystyrene sulfonates are preferably used as cationic ionic exchange resins.

For the purposes of controlled release, the active substances may also be present in a controlled release matrix. Preferably the active substance will be uniformly distributed in the matrix.

Physiologically compatible, hydrophilic materials, which are known to persons skilled in the art, may be used as matrix materials. Hydrophilic matrix materials which are used are preferably polymers, particularly preferably cellulose ethers, cellulose esters and/or acrylic resins. Especially preferred matrix materials include ethyl-cellulose, hydroxypropylmethylcellulose, hydroxypropyl-cellulose, hydroxymethylcellulose, poly(meth)acrylic acid and/or the derivatives thereof, such as the salts, amides or esters thereof.

Matrix materials prepared from hydrophobic materials, such as hydrophobic polymers, waxes, fats, long-chain fatty acids, fatty alcohols or corresponding esters or ethers or mixtures thereof are also preferred. Particularly preferably used hydrophobic materials include mono-or diglycerides of C12-C30 fatty acids and/or C12-C30 fatty alcohols and/or waxes or mixtures thereof.

It is also possible to use mixtures of the stated hydrophilic and hydrophobic materials as a controlled release matrix material.

In another preferred embodiment, the controlled release pharmaceutical formulations may also contain both the opioid and the α-agonist active substances in controlled release form.

The pharmaceutical formulation according to the invention may also contain at least one of the active substances both in controlled release form and in non-controlled release form. Combination with the immediately released active substance means that it is possible to achieve an elevated initial dose to alleviate pain rapidly. Slow release from the controlled release form then prevents the analgesic action from declining. Release of the active substances should particularly preferably be adjusted such that the controlled release pharmaceutical formulation need be administered at most twice, preferably just once daily. Persons skilled in the art know, based on the action of the analgesics, the mixing ratios in which they should be used in order to achieve the desired release of the active substances.

The pharmaceutical formulations according to the invention may moreover comprise still further coatings. Further coatings which may be present include those with pH-dependent dissolution behavior. It is thus possible to assure that the sub-units pass through the stomach in undissolved form and are released only when they reach the intestine. Coatings which serve to improve taste may also be used.

The pharmaceutical formulations according to the invention may be produced in accordance with various methods known to persons skilled in the art. Tablets, for example, may be produced by conventional processes such as, for example, extrusion, agglomerative build-up, wet granulation, fluidized bed processes, dry mixing, or compression molding processes. If the pharmaceutical formulation according to the invention, such as for example tablets, comprises coatings, these may be applied by conventional processes, such as for example sugar-coating, spray-application of solutions, dispersions or suspensions, by melt processes, or by powder application processes.

The quantity of active substance to be administered depends upon the active substances to be used and upon the route of administration. For oral administration, clonidine, for example, is preferably used in a quantity of between 1 μg and 500 μg, particularly preferably between 10 μg and 50 μg, in each case calculated relative to the base, and guanfacine is preferably used in a quantity of between 5 μg and 900 μg, particularly preferably between 100 μg and 500 μg, in each case calculated relative to the base.

If the combination is administered orally, morphine, for example, is preferably used in a quantity of between 0.1 mg and 20 mg, particularly preferably in a quantity of between 0.5 mg and 5 mg, in each case calculated relative to the base, and tramadol is preferably used in a quantity of between 1 mg and 50 mg, particularly preferably in a quantity of between 1 mg and 20 mg, in each case calculated relative to the base.

The pharmaceutical formulations according to the invention are preferably administered orally, parenterally or transdermally. It is particularly preferred to administer the formulations orally.

Transdermal controlled release formulations may, for example, be produced in the form of dressings having one or more active substance matrices or one or more active substance reservoirs and a control membrane.

Besides an opioid, an α-agonist and/or in each case the physiologically compatible salt thereof, the pharmaceutical formulations according to the invention may contain further pharmaceutical active substances and/or auxiliary substances. Useful pharmaceutical auxiliary substances include binders, extenders, lubricants, excipients, disintegration promoters, solvents, diluents, dyes, controlled release auxiliary substances and/or mixtures thereof. Selection of the auxiliary substances and the quantities thereof to be used are determined by whether the controlled release dosage forms according to the invention are used orally, parenterally or transdermally.

The term “extenders” is taken to mean, inter alia, starch, microcrystalline cellulose, dextrose, mannitol, or mixtures thereof.

Binders which may preferably be used include hydroxy-propylmethylcelluloses, polyvinylpyrrolidines, hydroxypropylcelluloses, starch paste, or mixtures thereof.

Disintegration promoters which are preferably used include hydroxypropylcelluloses having a low degree of substitution, crosspovidones, crosscarmelloses, starches, pectins, alginates, surfactants, or mixtures thereof.

Examples of useful lubricants include magnesium stearate, stearic acid, calcium stearate, fatty alcohols or mixtures thereof.

The present invention also provides a method of using the pharmaceutical formulations according to the invention for combating moderately severe to very severe pain. In comparison with using an opioid alone, the pharmaceutical formulations according to the invention exhibit a marked enhancement of analgesic action. This means that the quantity of opioid used may be distinctly reduced while the same analgesic action is achieved. Furthermore, the potential for opioid dependency and the constipating action of opioids may be distinctly reduced in comparison with using an opioid alone. This reduction in side-effects is still further enhanced because, due to the delayed release, only a relatively small quantity of the active substances is released at any one time. One particular advantage of the controlled release pharmaceutical formulations according to the invention is that the development of tolerance to the opioid is greatly delayed or completely avoided.

The following Examples are intended to illustrate the invention, but do not restrict the general concept or the scope of the invention.

### EXAMPLES

Granulation was performed in a Lödiger FM 5 high-speed mixer and tablets were produced using a Fette eccentric press.

For the purposes of the present invention, the term “PVP” should be taken to mean polyvinylpyrrolidones.

For the purposes of the present invention, the term “morphine HCl” means morphine HCl trihydrate.

For the purposes of the present invention, the term “tramadol HCl” means tramadol HCl trihydrate.

The term “min” means minutes.

The term “rpm” means revolutions per minute.

### Example 1

Production of Two-Layer Tablets With Controlled Release Opioid And Non-Controlled Release α-Agonist

| Constituent | Quantity per tablet in mg |
|---|---|
| Morphine HCl | 5.00 |
| Clonidine HCl | 0.30 |
| Lactose | 72.70 |
| Hydroxyethylcellulose | 11.00 |
| Cetostearyl alcohol | 33.00 |
| Talcum | 1.00 |
| Maize starch | 7.50 |
| PVP 30 | 2.00 |
| PVP Cl | 2.00 |
| Magnesium stearate | 0.88 |
| Total | 135.88 |

The two-layer tablets produced consisted of a controlled release layer containing the active substance morphine HCl and a non-controlled release layer containing the active substance clonidine. The controlled release granules were produced by processing morphine HCl, a proportion of the lactose, hydroxyethylcellulose and cetostearyl alcohol in a suitable mixer. The mixture was heated to 80° C. and granulated. After cooling, the granules were screened and mixed with magnesium stearate and talcum.

The non-controlled release granules were produced by granulating the remaining lactose and maize starch with a solution of clonidine HCl, PVP 30 and purified water in a suitable mixer. Magnesium stearate and PVP Cl were mixed into the dried granules. Both types of granules were compression molded to form the two-layer tablets.

In vitro release testing was performed in a paddle stirrer apparatus with a volume of 600 ml of dilute hydrochloric acid, at a pH of 1.2 and a speed of 75 rpm. Testing of the two layer tablet provided the following release profile over a period of 480 min (mean, n=6).

Release of morphine HCl

| Time in min | Quantity released in % |
|---|---|
| 0 | 0 |
| 30 | 31.5 |
| 60 | 44.9 |
| 180 | 80.1 |
| 300 | 97.4 |
| 480 | 100 |

Release of clonidine HCl

| Time in min | Quantity released in % |
|---|---|
| 0 | 0 |
| 5 | 53.3 |
| 10 | 94.9 |
| 15 | 100 |
| 30 | 100 |
| 60 | 100 |

### Example 2

Production of Two-Layer Tablets With Controlled Release Opioid And Non-Controlled Release α-Agonist

| Constituent | Quantity per tablet in mg |
|---|---|
| Morphine HCl | 5.00 |
| Clonidine HCl | 0.10 |
| Lactose | 72.90 |
| Hydroxyethylcellulose | 11.00 |
| Cetostearyl alcohol | 33.00 |
| Talcum | 1.00 |
| Maize starch | 7.50 |
| PVP 30 | 2.00 |
| PVP Cl | 2.00 |
| Magnesium stearate | 0.88 |
| Total | 135.88 |

The two-layer tablets produced consisted of a controlled release layer containing the active substance morphine HCl and a non-controlled release layer containing the active substance clonidine.

The controlled release granules were produced by processing morphine HCl a proportion of the lactose, hydroxyethylcellulose and cetostearyl alcohol in a suitable mixer. The mixture was heated to 80° C. and granulated. After cooling, the granules were screened and mixed with magnesium stearate and talcum.

The non-controlled release granules were produced by granulating the remaining lactose and maize starch with a solution of clonidine HCl, PVP 30 and purified water in a suitable mixer. Magnesium stearate and PVP Cl were mixed into the dried granules. Both types of granules were compression molded to form the two-layer tablets.

In vitro release testing was performed in a paddle stirrer apparatus with a volume of 600 ml of dilute hydrochloric acid, at a pH of 1.2 and a speed of 75 rpm. Testing of the two layer tablet provided the following release profile over a period of 480 min (mean, n=6).

Release of morphine HCl

| Time in min | Quantity released in % |
|---|---|
| 0 | 0 |
| 30 | 30.5 |
| 60 | 46.3 |
| 180 | 79.4 |
| 300 | 95.2 |
| 480 | 100 |

Release of clonidine HCl

| Time in min | Quantity released in % |
|---|---|
| 0 | 0 |
| 5 | 62.7 |
| 10 | 93.4 |
| 15 | 100 |
| 30 | 100 |
| 60 | 100 |

### Example 3

Production of Two-Layer Tablets With Controlled Release Opioid And Controlled Release α-Agonist

The two-layer tablets produced consisted of a controlled release layer containing the active substance tramadol HCl and another controlled release layer containing the active substance clonidine HCl.

Production of the first layer with tramadol HCl.

| Constituent | Quantity per tablet in mg |
|---|---|
| Tramadol HCl | 50.00 |
| Methylhydroxypropylcellulose | 80.00 |
| 100000 mPa*s | |
| Highly disperse silicon dioxide | 3.00 |
| Microcrystalline cellulose | 124.00 |
| Magnesium stearate | 3.00 |
| Total | 260.00 |

Tramadol HCl was mixed with microcrystalline cellulose, methylhydroxypropylcellulose, a proportion of the highly disperse silicon dioxide and magnesium stearate and precompressed to form tablets. The tablets were then broken, screened, and mixed with the remaining magnesium stearate and highly disperse silicon dioxide.

Production of the second layer with clonidine HCl.

| Constituent | Quantity per tablet in mg |
|---|---|
| Clonidine HCl | 0.30 |
| Lactose | 20.00 |
| Hydroxyethylcellulose | 11.00 |
| Cetostearyl alcohol | 33.00 |
| Talcum | 1.00 |
| Magnesium stearate | 0.70 |
| Total | 71.00 |

The lactose and hydroxyethylcellulose were initially introduced into a suitable mixer and mixed. The mixture was thoroughly moistened with a solution of clonidine HCl in water. After drying, the mixture was mixed with cetostearyl alcohol, heated to 80° C. and then granulated. The cooled granules were screened, combined with talcum and magnesium stearate, and the two types of granules were compression molded to form two-layer tablets.

In vitro release testing was performed in a paddle stirrer apparatus with a volume of 600 ml of dilute hydrochloric acid, at a pH of 1.2 and a speed of 75 rpm. Testing of the two layer tablet provided the following release profile over a period of 600 min (mean, n=6).

Release of tramadol HCl

| Time in min | Quantity released in % |
|---|---|
| 0 | 0 |
| 30 | 19.44 |
| 60 | 30.20 |
| 180 | 56.51 |
| 300 | 73.29 |
| 480 | 89.45 |
| 600 | 96.70 |

Release of clonidine HCl

| Time in min | Quantity released in % |
|---|---|
| 0 | 0 |
| 30 | 32.7 |
| 60 | 44.4 |
| 180 | 78.4 |
| 300 | 90.8 |
| 480 | 100 |
| 600 | 100 |

### Example 4

Production of A Two-Layer Tablet With Controlled Release Opioid And α-Agonist

The two-layer tablets produced consisted of a controlled release layer containing the active substance tramadol HCl and another controlled release layer containing the active substance clonidine HC1.

Production of the first layer with tramadol HCl.

| Constituent | Quantity per tablet in mg |
|---|---|
| Tramadol HCl | 50.00 |
| Methylhydroxypropylcellulose | 80.00 |
| 100000 mPa*s | |
| Highly disperse silicon dioxide | 3.00 |
| Microcrystalline cellulose | 124.00 |
| Magnesium stearate | 3.00 |
| Total | 260.00 |

Tramadol HCl was mixed with microcrystalline cellulose, methylhydroxypropylcellulose, a proportion of the highly disperse silicon dioxide and magnesium stearate and precompressed to form tablets. The tablets were then broken, screened, and mixed with the remaining magnesium stearate and highly disperse silicon dioxide.

Production of the second layer with clonidine HCl.

| Constituent | Quantity per tablet in mg |
|---|---|
| Clonidine HCl | 0.15 |
| Lactose | 20.15 |
| Hydroxyethylcellulose | 11.00 |
| Cetostearylcellulose | 33.00 |
| Talcum | 1.00 |
| Magnesium stearate | 0.70 |
| Total | 71.00 |

The lactose and hydroxyethylcellulose were initially introduced into a suitable mixer and mixed. The mixture was thoroughly moistened with an aqueous solution of clonidine HCl. After drying, the mixture was mixed with cetostearyl-cellulose, heated to 80° C. and then granulated. The cooled granules were screened, mixed with talcum and magnesium stearate, and the two types of granules were compression molded to form two-layer tablets.

In vitro release testing was performed in a paddle stirrer apparatus with a volume of 600 ml of dilute hydrochloric acid, at a pH of 1.2 and a speed of 75 rpm. Testing of the two layer tablet provided the following release profile over a period of 600 min (mean, n=6).

Release of tramadol HCl

| Time in min | Quantity released in % |
|---|---|
| 0 | 0 |
| 30 | 20.3 |
| 60 | 30.8 |
| 180 | 57.3 |
| 300 | 74.7 |
| 480 | 90.2 |
| 600 | 98.1 |

Release of clonidine HCl

| Time in min | Quantity released in % |
|---|---|
| 0 | 0 |
| 30 | 33.4 |
| 60 | 46.1 |
| 180 | 80.2 |
| 300 | 92.7 |
| 480 | 100 |
| 600 | 100 |

### Example 5

Production of Various Pellet Pharmaceutical Formulations

5.1 Rapid release active substance absorbed on a controlled release pellet

The active substance clonidine was applied as the α-agonist onto a controlled release morphine pellet using a suitable lacquer coating unit. The resulting pellets were packaged in capsules or compression molded to form tablets.

The controlled release pellets contained the following constituents:

| Constituent | Quantity per capsule in mg |
|---|---|
| Morphine sulfate | 10.00 |
| Lactose | 2.00 |
| Microgranules of sucrose and | 10.00 |
| maize starch USP 23-NF18 | |
| Polyethylene glycol 4000 | 2.50 |
| Ethylcellulose | 3.00 |
| Talcum | 0.15 |
| Dibutyl sebacate | 0.70 |
| Total | 26.35 |

Neutral starter nuclei were placed in the lacquer coating unit and moistened with an ethanolic polyethylene glycol 4000 solution. A mixture of morphine sulfate and lactose was repeatedly applied onto the moist nuclei, and the nuclei dried. This operation was repeated until the morphine sulfate/lactose mixture had been completely applied.

A suspension of clonidine HCl, hydroxypropyl-methylcellulose, polyethylene glycol 4000 and propylene glycol was applied onto the morphine pellets produced in this manner in a lacquer coating unit. The material applied had the following composition:

| Constituent | Quantity per capsule in mg |
|---|---|
| Clonidine HCl | 0.30 |
| Hydroxypropylmethylcellulose | 4.000 |
| Polyethylene glycol 4000 | 1.00 |
| Propylene glycol | 0.33 |
| Total | 26.35 |

The total quantity per capsule was 31.98 mg.

In vitro release testing was performed in a rotating basket apparatus with a volume of 600 ml of dilute hydrochloric acid and at a pH of 1.2 and a speed of 100 rpm. Testing of the formulation provided the following release profile over the period (mean, n=6).

Release of morphine sulfate

| Time in min | Quantity released in % |
|---|---|
| 0 | 0 |
| 60 | 28.5 |
| 180 | 34.3 |
| 240 | 46.2 |
| 430 | 64.4 |
| 600 | 81.1 |
| 720 | 98.5 |

Release of clonidine HCl

| Time in min | Quantity released in % |
|---|---|
| 0 | 0 |
| 5 | 50.3 |
| 10 | 93.9 |
| 15 | 100 |
| 30 | 100 |
| GO | 100 |

5.2 Mixed pellets in capsules

Production of tramadol pellets

| Constituent | Quantity per capsule in mg |
|---|---|
| Tramadol HCl | 50.00 |
| Hydroxypropylcellulose with a | 20.00 |
| low degree of substitution | |
| Microcrystalline cellulose | 106.00 |
| Calcium hydrogen phosphate | 20.00 |
| Hydroxypropylmethylcellulose | 4.00 |
| Aquacoat (ethylcellulose) | 20.00 |
| Dibutyl sebacate | 5.00 |
| Total | 225.00 |

Tramadol hydrochloride, microcrystalline cellulose, calcium hydrogen phosphate and the hydroxypropylcellulose with a low degree of substitution were thoroughly moistened with an aqueous solution of hydroxypropylmethylcellulose and extruded through a 0.5 mm perforated disk in a Pharmatex 35 T extruder. The extrudate was rounded in a Spheromat, dried in a fluidized bed, and then provided with a controlled release coating of an aqueous dispersion of ethylcellulose and dibutyl sebacate.

Production of clonidine pellets

| Constituent | Quantity per capsule in mg |
|---|---|
| Clonidine HCl | 0.30 |
| Microcrystalline cellulose | 120.00 |
| Hydroxypropylcellulose with a | 20.00 |
| low degree of substitution | |
| Hydroxypropylmethylcellulose | 4.00 |
| Total | 144.30 |

Microcrystalline cellulose and hydroxypropylcellulose with a low degree of substitution were thoroughly moistened with an aqueous solution of hydroxypropylmethylcellulose and clonidine HCl. The mixture was extruded through a 0.5 mm perforated disk in a Pharmatex 35 T extruder, rounded in a Spheromat, and dried in a fluidized bed. The coated tramadol and clonidine pellets were packaged in capsules and compression molded to form tablets.

In vitro release testing was carried out in a rotating basket apparatus with a volume of 600 ml of dilute hydrochloric acid, at a pH of 1.2 and a speed of 100 rpm. Testing of the capsules provided the following release profile over the period (mean, n=6).

Release of tramadol HCl

| Time in min | Quantity released in % |
|---|---|
| 0 | 0 |
| 120 | 13.0 |
| 240 | 31.0 |
| 480 | 57.0 |
| 600 | 71.0 |
| 720 | 100 |

Release of clonidine HCl

| Time in min | Quantity released in % |
|---|---|
| 0 | 0 |
| 5 | 75.1 |
| 10 | 96.3 |
| 15 | 96.8 |
| 30 | 96.9 |
| 60 | 97.0 |

### Example 6

Matrix tablets having the following composition

| Constituent | Quantity per tablet in mg |
|---|---|
| Morphine HCl | 5.00 |
| Clonidine HCl | 0.30 |
| Lactose | 20.00 |
| Hydroxyethylcellulose | 11.00 |
| Cetostearyl alcohol | 33.00 |
| Talcum | 1.00 |
| Magnesium stearate | 0.70 |
| Total | 71.00 |

Morphine HCl, lactose, hydroxyethylcellulose and cetostearyl alcohol were mixed, and the resulting mixture was thoroughly moistened with aqueous clonidine HCl. The mixture was dried, then heated to 80° C. and granulated. After cooling, the granules were screened, mixed with magnesium stearate, and tableted.

In vitro release testing was performed in a paddle stirrer apparatus with a volume of 600 ml of dilute hydrochloric acid, at a pH of 1.2 and a speed of 75 rpm. Testing of the matrix tablet provided the following release profile over a period of 480 min (mean, n=6).

Release of morphine HCl

| Time in min | Quantity released in % |
|---|---|
| 0 | 0 |
| 30 | 31.5 |
| 60 | 44.9 |
| 180 | 80.1 |
| 300 | 97.4 |
| 480 | 100 |

Release of clonidine HCl

| Time in min | Quantity released in % |
|---|---|
| 0 | 0 |
| 30 | 32.7 |
| 60 | 44.4 |
| 180 | 78.4 |
| 300 | 90.8 |
| 480 | 100 |

### Example 7

Matrix tablets having the following composition

| Constituent | Quantity per tablet in mg |
|---|---|
| Tramadol HCl | 50.0 |
| Clonidine HCl | 0.20 |
| Methylhydroxypropylcellulose, | 85.00 |
| type 2208, 100000 mPa * s | |
| Highly disperse silicon dioxide | 5.00 |
| Calcium hydrogen phosphate | 155.80 |
| Magnesium stearate | 4.00 |
| Total | 300.00 |

The total quantity of starting materials was 200 g. The constituents were screened (0.63 mm), then mixed for 10 minutes in a small cube mixer and compression molded in a Korsch EK 0 eccentric tablet press to form 10 mm diameter tablets with a radius of curvature of 8.5 mm and an average weight of 300 mg.

In vitro release testing was performed in a paddle stirrer apparatus with a volume of 600 ml of dilute hydrochloric acid, at a pH of 1.2 and a speed of 75 rpm. Testing of the matrix tablet provided the following release profile over a period of 480 min (mean, n=6).

Release of tramadol HCl

| Time in min | Quantity released in % |
|---|---|
| 0 | 0 |
| 30 | 22.6 |
| 60 | 35.2 |
| 180 | 52.4 |
| 300 | 78.2 |
| 480 | 86.3 |

Release of clonidine HCl

| Time in min | Quantity released in % |
|---|---|
| 0 | 0 |
| 30 | 23.2 |
| 60 | 36.8 |
| 180 | 51.3 |
| 300 | 79.2 |
| 480 | 87.7 |

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

What is claimed is:

1. A pharmaceutical formulation comprising in combination:
an opioid or physiologically compatible salt thereof, and
an α-agonist or physiologically compatible salt thereof, wherein at least one of said opioid and α-agonist is present in delayed release form.

2. A pharmaceutical formulation as claimed in claim 1, wherein the opioid is present in delayed release form.

3. A pharmaceutical formulation as claimed in claim 2, wherein the opioid is released over a period of at least 8 hours.

4. A pharmaceutical formulation as claimed in claim 3, wherein the opioid is released over a period of 12 hours.

5. A pharmaceutical formulation as claimed in claim 3, wherein the opioid is release over a period of 24 hours.

6. A pharmaceutical formulation as claimed in claim 1, wherein both the opioid and the α-agonist are present in delayed release form.

7. A pharmaceutical formulation as claimed in claim 1, wherein the opioid comprises at least one compound selected from the group consisting of morphine, hydromorphone, codeine, oxycodone, dihydrocodeine, dextropropoxyphene, buprenorphine, levomethadone, fentanyl, sufentanil, etorphine, pentazocine, tilidine, tramadol, levorphanol, methadone, dihydromorphine, pethidine, piritramide, and physiologically compatible salts of any of the foregoing.

8. A pharmaceutical formulation as claimed in claim 7, wherein the opioid is selected from the group consisting of morphine, tramadol and physiologically compatible salts thereof.

9. A pharmaceutical formulation as claimed in claim 1, wherein the α-agonist comprises at least one compound selected from the group consisting of clonidine, guanfacine, guanabenz, lofexidine, adrenaline, methyldopa, noradrenaline, methoxamine, oxymetazoline, xylometazoline, teryzoline, ST-91, medetomidine, dexmedetomidine, agmatine, UK14, 304, paraaminoclonidine, U-47, 476A, DJ-741, ICI-106270, xylazine, talipexole (BHT-920), naphazoline, tizanidine, and physiologically compatible salts of any of the foregoing.

10. A pharmaceutical formulation as claimed in claim 9, wherein the α-agonist is selected from the group consisting of clonidine, guanfacine and physiologically compatible salts thereof.

11. A pharmaceutical formulation as claimed in claim 1, wherein the opioid and u-agonist, respectively, are present in a weight ratio of opioid to α-agonist in the range from 200:1 to 10:1.

12. A pharmaceutical formulation as claimed in claim 11, wherein the ratio of opioid to α-agonist is in the range from 100:1 to 10:1.

13. A pharmaceutical formulation as claimed in claim 1, in the form of a tablet or capsule.

14. A pharmaceutical formulation as claimed in claim 13, in the form of a sugar-coated tablet.

15. A pharmaceutical formulation as claimed in claim 13, in the form of a multilayer tablet.

16. A pharmaceutical formulation as claimed claim 1, wherein said formulation is in multi-particulate form.

17. A pharmaceutical formulation as claimed in claim 16, wherein said multi-particulate form is selected from the group consisting of microtablets, microcapsules, ion exchange resinates, granules, active substance crystals, and pellets.

18. A pharmaceutical formulation as claimed in claim 1, wherein said delayed release form comprises a controlled release coating, immobilization on an ion exchange resin, embedding in a controlled release matrix, or a combination at least two of the foregoing.

19. A pharmaceutical formulation as claimed in claim 18, wherein said delayed release form comprises a coating composed of a water-insoluble polymer or wax.

20. A pharmaceutical formulation as claimed in claim 19, wherein said coating comprises a water-insoluble polyacrylic resin or cellulose derivative.

21. A pharmaceutical formulation as claimed in claim 20, wherein said coating comprises a water-insoluble alkylcellulose.

22. A pharmaceutical formulation as claimed in claim 20, wherein said coating comprises a water-insoluble ethylcellulose or poly(meth)acrylate polymer.

23. A pharmaceutical formulation as claimed in claim 18, wherein said delayed release form comprises a controlled release matrix comprising at least one substance selected from the group consisting of polymers, waxes, fats, fatty acids, fatty alcohols, and fatty acid or fatty alcohol esters or ethers.

24. A pharmaceutical formulation as claimed in claim 23, wherein said controlled release matrix comprises at least one polymeric substance selected from the group consisting of cellulose ethers, cellulose esters and acrylic resins.

25. A pharmaceutical formulation as claimed in claim 23, wherein said controlled release matrix comprises at least one substance selected from the group consisting of ethylcellulose, hydroxyethylcellulose, hydroxypropylcellulose, hydroxypropylmethylcellulose, mono-and diglycerides of C12 to C30 fatty acids, C12-C30 fatty alcohols, and mixtures of two or more of the foregoing.

26. A pharmaceutical formulation as claimed in claim 1, wherein at least one of said opioid and said α-agonist is present in both controlled release form and non-controlled release form.

27. A pharmaceutical formulation as claimed in claim 1, wherein said formulation is administrable orally, parenterally or transdermally.

28. A pharmaceutical formulation as claimed in claim 1, wherein said formulation is orally administrable.

29. A method of treating a patient suffering from a pain condition, said method comprising administering to said patient an effective pain relieving amount of a pharmaceutical formulation as claimed in claim 1.

30. A method as claimed in claim 29, wherein said pain condition is a moderately severe to severe acute or chronic pain state.
